(19) United States
(12) Patent Application Publication
(10) Pub. No.: US 2024/0362834 A1
(43) Pub. Date: Oct. 31, 2024

(54) X-RAY CT APPARATUS AND IMAGING SUPPORT METHOD

(71) Applicant: FUJIFILM Healthcare Corporation, Kashiwa-shi (JP)

(72) Inventors: Yusuke Tetsumura, Chiba (JP); Ryota Kohara, Chiba (JP)

(21) Appl. No.: 18/643,448

(22) Filed: Apr. 23, 2024

(30) Foreign Application Priority Data
Apr. 27, 2023 (JP) 2023-072998

(51) Int. Cl. G06T 11/00; A61B 6/03; G06T 5/50; G06T 7/00; G06T 7/20

(52) U.S. Cl. CPC G06T 11/005; A61B 6/032; G06T 5/50; G06T 7/0014; G06T 7/20; G06T 11/008; G06T 2207/10081; G06T 2207/10116; G06T 2207/20104; G06T 2207/30048; G06T 2207/30168; G06T 2207/30196; G06T 2210/41

(57) ABSTRACT

A center phase (image reconstruction phase) of reconstruction can be appropriately set even in a case in which motion correction is applied to reconstruct an image, to ensure a good image quality, and to support optimum phase search work by a doctor, an examination technician, or the like (hereinafter, referred to as an operator). An arithmetic operation unit that performs image reconstruction in an X-ray CT apparatus includes a motion correction reconstruction unit that detects motion information of a subject and performs motion correction reconstruction, and an image quality score calculation unit that calculates an image quality score for evaluating an image quality in one or a plurality of motion phases with respect to an image reconstructed by the motion correction reconstruction unit. An interface that can perform a phase search while comparing motion correction using the displayed image quality score is provided.

### CROSS-REFERENCE TO RELATED APPLICATION

The present application claims priority from Japanese Patent Application No. 2023-072998, filed on Apr. 27, 2023, the content of which is hereby incorporated by reference into this application.

### BACKGROUND OF THE INVENTION

### 1. Field of the Invention

The present invention relates to an X-ray CT apparatus, and particularly relates to a support technique for determining an optimum motion phase for imaging in a case of imaging a target having motion or movement, such as a heart.

### 2. Description of the Related Art

In an X-ray CT apparatus, an X-ray source and an X-ray detector mounted on a scanner are disposed to face each other with a subject as a center, and continuously imaging is performed while rotating the scanner and moving the scanner along a body axis direction of the subject, to acquire 3D image data of the subject including an imaging target part. In a case in which the target part is a moving target such as a heart, the imaging and the image reconstruction are performed using a motion phase having the least motion as a target. For example, in cardiac imaging, out of a systolic phase and a diastolic phase of the heart, a cardiac phase in the diastolic phase in which the motion is relatively little is set as an imaging center phase or an image reconstruction phase. In the cardiac imaging, it is important to set the cardiac phase during the imaging and the reconstruction as an optimum phase in order to obtain a CT image with a good image quality, and a method of determining the optimum phase is proposed (WO2012/176745A and JP2019-208752A).

For example, WO2012/176745A proposes a technique of acquiring a plurality of tomographic data in different cardiac phases, calculating an amount of variation between the cardiac phases from the plurality of tomographic data, and obtaining a phase (cardiac phase) in which the motions of the respective parts of the heart are in harmony as an optimum cardiac phase. JP2019-208752A proposes a method of estimating a timing of a stationary phase from a change of a contrast medium in a plurality of temporal images by using the contrast medium.

### SUMMARY OF THE INVENTION

In CT, in order to reconstruct image data of one cross section, it is necessary to rotate the scanner by 180 degrees or more, which causes a decrease in image quality due to the motion of the subject during the rotation. On the other hand, a technique (motion correction reconstruction) of detecting the motion during the imaging and correcting the motion to reconstruct an image is developed. In the motion correction reconstruction, for example, the motion is detected by using projection data (image pair data) in a predetermined range about an angle of ±90 degrees with respect to the image reconstruction phase, and the reconstruction is performed while correcting the motion.

In such motion correction reconstruction, a position of the optimum cardiac phase set in a case in which the motion correction is not applied may be changed due to factors such as an imaging condition or the motion of the imaging part, and there is a possibility that sufficient accuracy cannot be obtained in the setting of the optimum cardiac phase on the premise that there is no motion correction. In a case in which the optimum phase is to be set in consideration of the motion correction, the work may be complicated and the work cost may be increased.

An object of the present invention is to provide a technique capable of ensuring a good image quality even in a case in which motion correction is applied. Another object of the present invention is to provide a support technique of facilitating optimum phase search work by a doctor, an examination technician, or the like (hereinafter, referred to as an operator).

An aspect of the present invention for solving the above-described problems is to provide a unit that calculates an image quality score in a case in which the motion correction is applied, as a function of the X-ray CT apparatus. The image quality score is an indicator for evaluating the image quality of the image obtained in a case in which the motion correction is applied, and is information for setting the optimum phase.

Another aspect of the present invention is to provide a unit (interface) that presents information (optimum phase determination auxiliary information) for optimum phase determination including the image quality score, as a function of the X-ray CT apparatus. The operator who determines the optimum phase can perform a phase search while comparing the motion correction based on the information presented through the interface, and can improve the efficiency of the optimum phase search work.

Specifically, an aspect of the present invention relates to an X-ray CT apparatus comprising: a scanner including an X-ray source and an X-ray detector, which are disposed to face each other, and rotating around a subject; and an arithmetic operation unit that generates a tomographic image of the subject by using X-ray transmission data detected by the X-ray detector, in which the arithmetic operation unit includes a motion correction reconstruction unit that detects motion information of the subject and performs motion correction reconstruction, and an image quality score calculation unit that calculates an image quality score for evaluating an image quality in one or a plurality of motion phases with respect to an image reconstructed by the motion correction reconstruction unit.

Another aspect of the present invention relates to an imaging support method of supporting search work for an image reconstruction phase during cardiac imaging using an X-ray CT apparatus, the imaging support method including: a step (1) of setting a phase range in which the image reconstruction phase is searched for by using motion information of a subject; and a step (5) of searching for the range and determining the image reconstruction phase, in which, after the step (1), a step (2) of calculating an image quality score for evaluating an image quality in a case in which motion correction reconstruction is performed for each phase included in the phase range, a step (3) of presenting the image quality score to an operator, and a step (4) of receiving determination of the image reconstruction phase from the operator are included.

According to the aspects of the present invention, in a case of reconstructing the CT image in the optimum phase of the motion, the image quality score in a case in which the motion correction is applied is calculated, so that the optimum phase search accuracy can be improved by using the image quality score. In addition, the auxiliary information including the image quality score for the optimum phase search is presented to the operator, so that the operator can easily perform the optimum phase search work, and can obtain a good image to which the motion correction is applied.

### BRIEF DESCRIPTION OF THE DRAWINGS

FIG. 1 is a diagram showing an overall outline of an X-ray CT apparatus to which the present invention is applied.

FIG. 2 is a functional block diagram of the X-ray CT apparatus.

FIG. 3 is a flowchart showing an outline of an operation of the X-ray CT apparatus according to the embodiment of the present invention.

FIG. 4 is a functional block diagram of an image processing unit according to Embodiment 1.

FIG. 5A is a diagram showing a part of processing according to Embodiment 1 of the present invention.

FIG. 5B is a diagram showing a part of the processing according to Embodiment 1 of the present invention.

FIG. 6A is a diagram showing search phase setting processing according to Embodiment 1 of the present invention.

FIG. 6B is a diagram showing the search phase setting processing according to Embodiment 1 of the present invention.

FIG. 7 is a diagram showing a flow of image quality score calculation processing according to Embodiment 1 of the present invention.

FIG. 8 is a diagram showing the image quality score calculation processing.

FIG. 9 is a diagram showing a display screen example according to Embodiment 2.

FIG. 10 is a diagram showing an example of a processing flow according to Embodiment 2.

### DESCRIPTION OF THE PREFERRED EMBODIMENTS

Embodiments of the present invention will be described below with reference to the drawings.

First, an overall configuration of an X-ray CT apparatus to which the present invention is applied will be described.

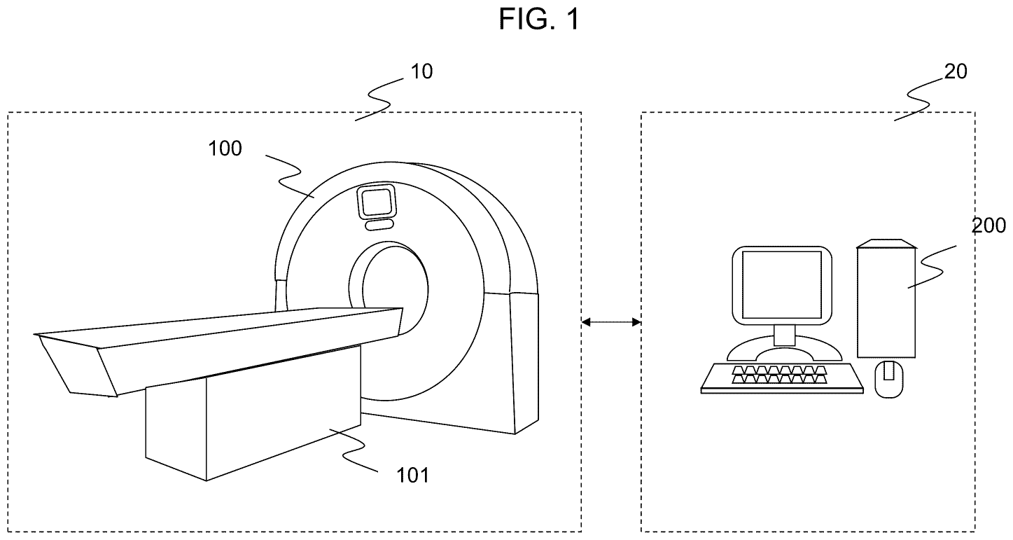

As shown in FIG. 1, the X-ray CT apparatus 1 comprises an imaging unit 10 comprising a gantry 100 for capturing a tomographic image and a fluoroscopic image of a subject and a bed device 101 on which the subject lies down, and an operation unit 20 that operates and controls the imaging unit 10.

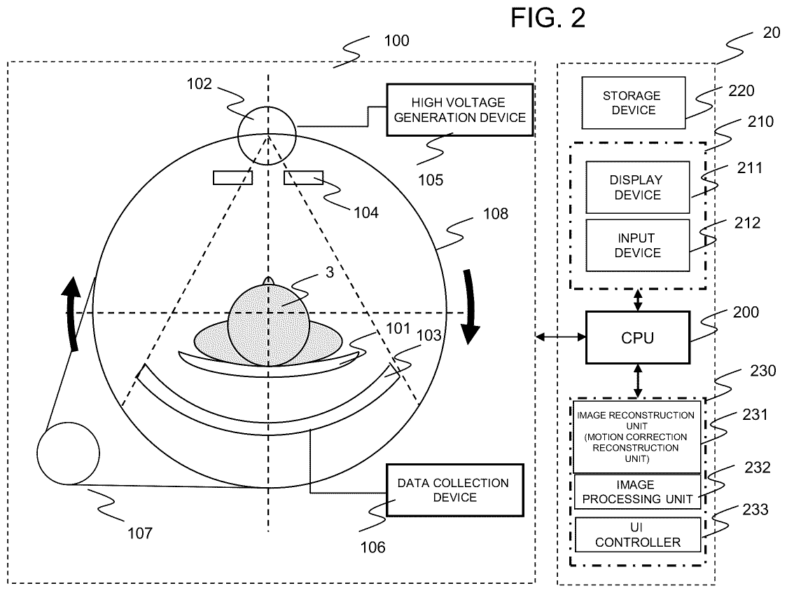

As shown in FIG. 2, the gantry 100 comprises an X-ray generation device 102 that generates X-rays with which a subject 3 is irradiated, a collimator device 104 that narrows a flux of the X-rays generated from the X-ray generation device 102, an X-ray detection device 103 that detects the X-rays transmitted through the subject, a scanner 108 on which the X-ray generation device 102, the collimator device 104, and the X-ray detection device 103 are mounted, a high voltage generation device 105 that applies a high voltage to the X-ray generation device 102, a data collection device 106 that collects transmitted X-ray data obtained from the X-ray detection device 103, and a drive device 107 that rotates the scanner 108 around the subject 3. The X-ray generation device 102 comprises an X-ray tube (not shown), and the subject 3 is irradiated with a predetermined amount of the X-rays by a predetermined tube current flowing through the X-ray tube.

The operation unit 20 comprises a central processing unit (CPU) 200 that controls each device built in the gantry, a user interface unit (UI unit) 210 that is used to interact with a user and the central processing unit 200, and a storage device 220 that stores programs, device parameters, or the like executed by the CPU 200 and data necessary for imaging. An arithmetic operation unit 230 that performs various arithmetic operations such as image reconstruction on the transmitted X-ray data collected by the data collection device 106 is mounted in the central processing unit 200.

The CPU 200 controls, in response to an operation instruction from an operator via an input device 212, the imaging unit 10 (X-ray generation device 102, X-ray detection device 103, high voltage generation device 105, collimator device 104, bed device 101, drive device 107, data collection device 106), the UI unit 210, and an arithmetic operation unit 230. Each of these units operates under the control of the central processing unit 200 to reconstruct a CT image, correct the reconstructed CT image, and the like.

The functions of the CPU 200 are realized by the central processing unit 200 reading and executing a program in which an arithmetic operation algorithm or a processing procedure of control is described. It should be noted that a part of the arithmetic operation or the processing performed by the arithmetic operation unit 230 may be performed by using a programmable logic device (PLD), such as an ASIC or an FPGA.

The UI unit 210 includes the input device 212 for the operator to input an imaging condition and the like, and a display device 211 that displays data, such as a captured image, and a GUI.

The arithmetic operation unit 230 includes an image reconstruction unit 231 that performs back projection processing on the transmitted X-ray data obtained by the data collection device 106 to create the tomographic image, an image processing unit 232 that performs processing of determining an optimum phase in analysis of image data or cardiac imaging, and a UI controller 233 that controls the UI unit 210. The image reconstruction performed by the image reconstruction unit 231 includes motion-corrected image reconstruction to which motion correction of the subject is applied.

The X-ray CT apparatus according to the present embodiment has a function of performing imaging a moving part as an imaging target, such as cardiac imaging, performing the image reconstruction by setting, as a center phase, a scanner angle at which a motion phase of the subject is a phase at which an optimum image quality is obtained (optimum image reconstruction phase, also simply referred to as an optimum phase), and displaying an image of the optimum phase.

Therefore, the image processing unit 232 has a function of searching for and setting the optimum phase, a function of evaluating the image quality in a case in which the motion correction is applied to a predetermined phase range for searching for the optimum phase (image quality score calculation function), a function of generating a partial tomographic image pair necessary for the motion correction, and the like. The partial tomographic image pair is a pair of tomographic images reconstructed by using X-ray projection data in a predetermined angle range about an angle (180 degrees) at which the rotation angles of the scanner are located at positions facing each other, respectively, and is a partial tomographic image because the image is reconstructed by using the projection data in which the predetermined angle range is less than 180 degrees. Such a partial tomographic image pair can be used in a case of detecting and correcting the motion in the motion correction reconstruction, and can also be used in the processing of determining the optimum phase in the present embodiment.

In addition, in the preferred embodiment, information generated by the image processing unit 232 is presented to the operator through the UI controller 233 and the UI unit 210, and a function of searching for and setting the optimum phase is realized in an interactive manner with the operator. The image reconstruction unit 23 1 performs the image reconstruction to which the motion correction is applied or not applied by using the processing result of the image processing unit 232.

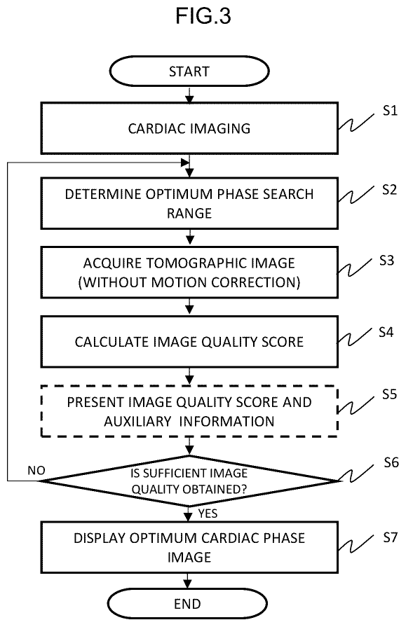

Hereinafter, a flow of an operation of the X-ray CT apparatus according to the present embodiment including the processing of the image processing unit 232 will be described with reference to a case of the cardiac imaging. An outline of the operation is shown in FIG. 3.

First, the imaging unit 10 performs the cardiac imaging in response to a command from the operation unit 20 (S1). Specifically, the scanner 108 is rotated to perform imaging while the bed device 101 on which the subject is placed in a direction orthogonal to a rotation surface of the scanner 108 is moved. Biological information such as an electrocardiogram of the subject is acquired simultaneously with this imaging. In a case in which the imaging condition is set before the imaging is started, the image reconstruction phase may be set as a temporary image reconstruction phase in the diastolic phase with a relatively small motion from a cardiac period obtained from the electrocardiogram information. In this case, the tube current supplied to the X-ray tube in a predetermined time width including the image reconstruction phase may be set to be high, so that a signal with good SN can be obtained.

By this imaging, the data collection device 106 collects the transmitted X-ray data of a plurality of cross sections along a body axis of the subject.

Then, the image processing unit 232 determines a search phase for searching for the optimum image reconstruction phase (S2). In this step S2, as the search phases, a plurality of phases may be individually determined, or a predetermined phase range may be set. As described above, the optimum image reconstruction phase is a phase of a relatively stable cardiac period in which the motion of the heart, which is the imaging target, is stable, and in a case in which the image reconstruction phase is set as the imaging condition, the setting can be easily and automatically performed on the apparatus side using the information, or based on the tube current information or the electrocardiogram information.

The image reconstruction unit 231 reconstructs the tomographic images of the plurality of cross sections using the projected X-ray data of 180 degrees or more acquired in step S1 (S3). This reconstruction is general reconstruction (for example, filtered back projection, iterative reconstruction, and the like) in which the motion correction is not applied. In this case, the partial tomographic image pair may be generated, and the reconstruction (motion correction reconstruction) to which the motion correction is applied may be performed. The processing via the image reconstruction unit 231 may be executed in parallel with the processing (step S2) via the image processing unit 232.

The image processing unit 232 evaluates the image quality of the tomographic images obtained in step S3 to calculate an image quality score (S4). The image quality is evaluated using the image itself or the tube current information in a case of the image acquisition for at least one of the tomographic images to which the motion correction is not applied or the tomographic image to which the motion correction is applied.

The UI controller 233 presents the calculated image quality score to the display device 211 together with other auxiliary information (S5). An aspect of the presentation will be described in detail in the embodiment described below, but the image quality score for each phase is displayed together with a GUI or the like that receives the tomographic image or an operator's operation. The GUI can include, for example, display of receiving a change of the search phase or the confirmation of the optimum phase. The operator can change the search phase or confirm the optimum phase by checking the presented image quality score.

In a case in which the operator determines that the image quality is sufficient and operates the GUI for confirming the optimum phase (S6), the image processing unit 232 displays the tomographic image of the optimum phase generated by the image reconstruction unit 231 (S7). The generation of the tomographic image of the optimum phase can be performed after the optimum phase is confirmed, but the image reconstruction unit 231 can generate the tomographic image for the phase in the search range in the background of the processing of the image processing unit 232, and the tomographic image can be immediately displayed at the time when the optimum phase is confirmed, which is suitable.

On the other hand, in a case in which it is determined in step S6 that the sufficient image quality is not obtained after the auxiliary information is presented and the search phase is changed, the processing returns to step S2. The image processing unit 232 changes the search phase by reflecting the change made by the operator. The following processing (S3 to S5) is repeated.

As a result of such a repetition, the tomographic image (motion correction reconstruction image or general reconstruction image) is displayed in the optimum phase determined by the operator to have a sufficient image quality. The number of repetitions is not limited in the flow shown in FIG. 3, but may be appropriately limited, and processing of returning to the imaging (S1) or the like again may be added in a case in which a sufficient image quality is not obtained.

In the above description, the operator determines the optimum phase after the image quality score is calculated, but the apparatus side can determine the optimum phase based on the image quality score. In this case, step S5 is omitted.

As described above, with the X-ray CT apparatus according to the present embodiment, for each phase of the search range for the optimum phase, the image quality in a case in which the phases are set as the center phase is estimated, and the image quality score is calculated, so that it is possible to easily determine the optimum phase. By presenting the image quality score for each phase, the operator can easily change the search phase or confirm the optimum phase by using the image quality score as the support information.

Hereinafter, embodiments of specific processing performed by the arithmetic operation unit 230 of the X-ray CT apparatus will be described. In the following embodiments, FIGS. 1 to 3 used in the description of the above embodiment are common, and the description will be made with reference to these drawings as appropriate.

### Embodiment 1

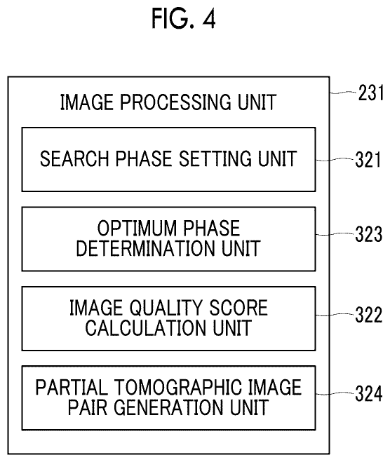

In the present embodiment, the image processing unit 232 calculates the image quality score using the partial tomographic image pair. FIG. 4 is a functional block diagram of the image processing unit 232 according to the present embodiment.

As shown in FIG. 4, the image processing unit 232 according to the present embodiment comprises a search phase setting unit 321 that sets the phase range for searching for the optimum phase, an image quality score calculation unit 322 that calculates the image quality scores of the plurality of phases as information used for the search processing for the optimum phase, and an optimum phase determination unit 323 that determines the optimum image reconstruction phase based on the image quality score. Further, a partial tomographic image pair generation unit 324 that generates the partial tomographic image pair used for the calculation of the image quality score and the motion correction is provided.

Hereinafter, the operation of the X-ray CT apparatus according to the present embodiment will be described mainly focusing on the processing of the image processing unit 232. A flow of the operation of the X-ray CT apparatus is the same as the flow shown in FIG. 3, and the following description will be made with reference to FIG. 3 as appropriate.

Imaging and Search for Optimum Phase: S1 and S2

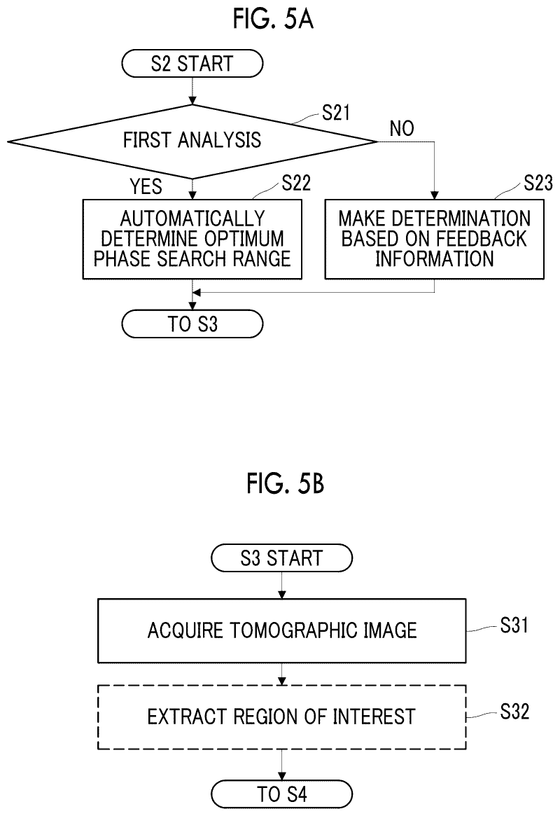

In a case in which the cardiac imaging is performed and the data collection device 106 collects the transmitted X-ray data of the plurality of cross sections along the body axis of the subject (S1), the processing for the optimum phase search is started (S2). In this processing, the phase range in which the search is performed (search phase range) for determining the final phase is set. Specifically, as shown in FIG. 5A, in the first processing, the search target phase is determined by the simple automatic analysis based on at least one or more data of the tube current information or the electrocardiogram information.

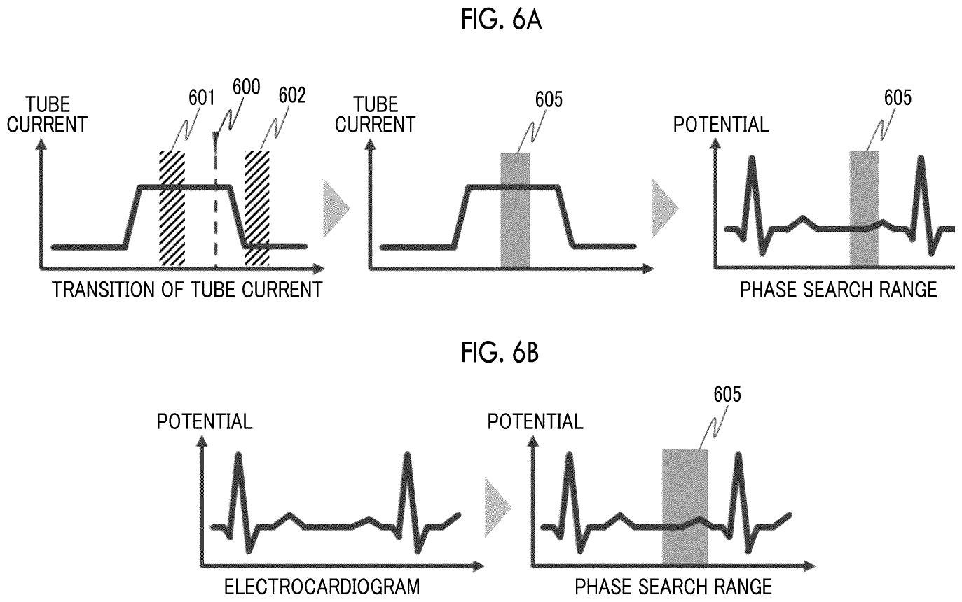

The determination of the search phase based on the tube current is performed by using the tube current information in the imaging in a case in which the tube current is set such that the tube current is increased in a stationary phase such as a diastolic phase based on the electrocardiogram information to perform the imaging. As an example of the method using the tube current information, an average tube current in a case in which the partial image pair at angles facing each other about a phase of interest is acquired is used. That is, as shown in FIG. 6A, a ratio of the average of the tube current (average tube current) in a case in which projection data 601 and 602 in the predetermined angle range constituting the partial image pair are acquired is calculated. In a case in which the value of the average tube current ratio of the partial image pair for a phase of interest 600 is a phase close to 1 (for example, 1±10%), the phase of interest is set as a search phase 605. Instead of the calculation of the ratio of the tube current, a phase of which the average tube current of the facing partial image pair about the phase of interest is equal to or higher than a certain value may be automatically selected. As a result, as shown on the right side of FIG. 6A, a range in which it is estimated that the high-quality motion- corrected image can be acquired is automatically set.

In the method using the electrocardiogram information, as shown in FIG. 6B, the range of the cardiac phase in which the motion is stable is empirically set based on the heart rate

(HR). Specifically, for example, the diastolic middle stage is automatically selected in a case in which HR≤65, the diastolic middle stage and the systolic end stage are automatically selected in a case in which 65<HR<85, and the systolic end stage is automatically selected in a case in which 85≤HR. The determination of the search phase may be performed by only one of the method using the tube current or the method using the electrocardiogram information, or may be performed by combining both methods. In a case of combining the methods, for example, in a case in which the search phase range is too wide in one method, AND with the other method may be used, and conversely, in a case in which the search phase range is too narrow, OR in both methods may be used.

The above-described processing is the setting processing of the first search phase, and the second processing and subsequent processing are the processing of reflecting the subsequent processing result (S23).

Acquisition of Tomographic Image and Calculation of Image Quality Score: S3 and S4

In a case in which the search range for the phase is determined, the image quality score is calculated (S4). The image quality score may be calculated for the reconstructed tomographic image (S3) without performing the motion correction or may be calculated for the tomographic image subjected to the motion correction reconstruction. The image quality score may be calculated for the entire tomographic image or may be calculated only for a region of interest as shown in FIG. 5B. In a case in which the image quality score is calculated for the region of interest, from a general reconstruction image (tomographic image) generated from the projection data of 180 degrees or more (S31), a region (for example, a heart or a coronary artery) near a diagnosis target is extracted as the region of interest by using a method such as filtering or deep learning (S32).

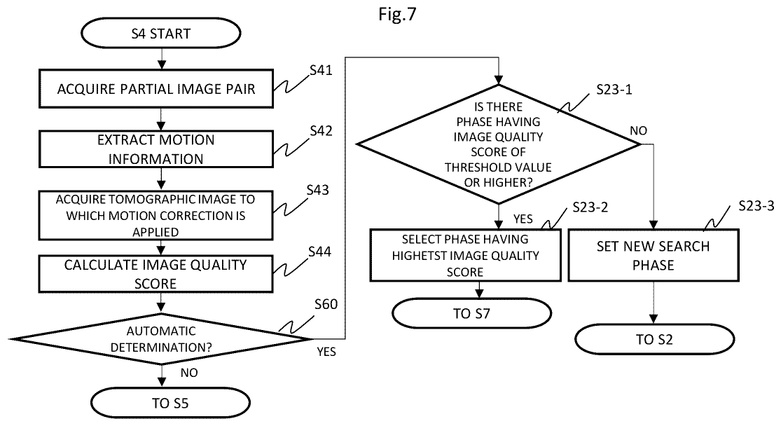

Hereinafter, a calculation example of a case in which the image quality score is calculated for the tomographic image subjected to the motion correction reconstruction will be described. In a case of performing the motion correction reconstruction, as shown in FIG. 7, the calculation of the image quality score includes the acquisition of the partial image pair (S41), the generation of the tomographic image to which the motion correction is applied (S42), and the calculation of the image quality score for the tomographic image to which the motion correction is applied (S43), and S41 to S43 can be performed at any timing after the processing (S3) of acquiring the tomographic image to which the motion correction is not applied is performed, as long as the execution is performed in this order.

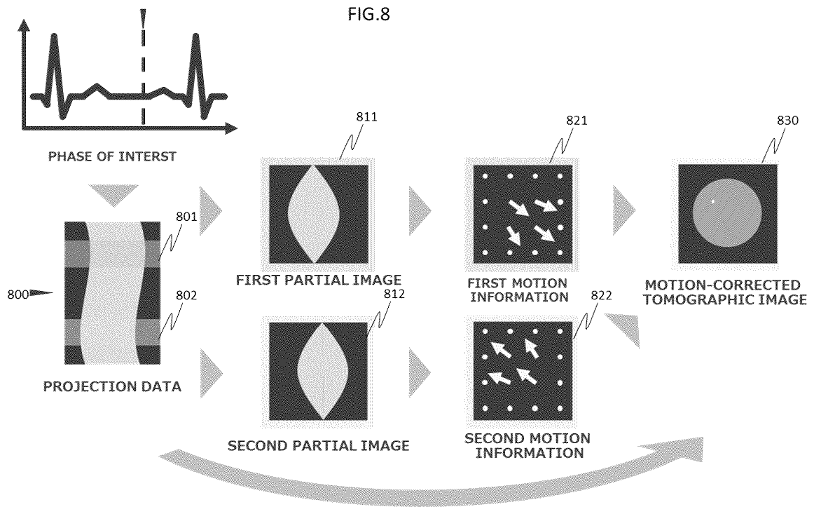

First, the facing partial image pair about the phase of interest is acquired (S41). As shown in FIG. 8, the facing partial image pair is a pair of partial images (first partial image 811 and second partial image 812) obtained by extracting partial regions 801 and 802 facing each other by about 60° about a phase of interest 800 from the projection data, and performing the back projection. It is preferable that the partial image pair is subjected to filtering using, for example, a smoothing filter such as a bilateral filter for noise removal.

Next, the motion information is acquired from the facing partial image pair (S42). The motion information is acquired as motion information (vector map) 821 and 822 by performing registration on the facing partial image pair. In a case in which the region of interest is extracted to calculate the image quality score, the score may be normalized in the related processing in a case in which the size of the region of interest is different for each slice.

As the registration, a known method such as a method based on a B-spline function or a method based on an optical flow can be used. In order to increase the speed of the registration, the registration may be performed in two dimensions or may be performed in three or four dimensions by creating a plurality of images. The number of pixels to be subjected to the registration may be down-sampled to ¼ or 1/16. Further, for the registration, a simple and light method for the phase search may be used instead of a high-accuracy and heavy method used for the actual motion correction reconstruction.

After calculating the motion information 821 and 822, the tomographic image 830 to which the motion correction is applied is acquired (S43). Specifically, the motion-corrected tomographic image is acquired by performing the back projection on the projection data while considering the motion information. In this case as well, the number of pixels of the tomographic image may be down-sampled to ¼ or 1/16, which makes it possible to increase the speed of the motion correction reconstruction in the calculation of the image quality score.

Next, the image quality score of the image to which the motion correction is applied or not applied is calculated by using one or more information acquired in steps S41 to S43 (S44).

### Calculation Using Facing Partial Image Pair

As the image quality score using the facing partial image pair, an evaluation indicator indicating a degree of similarity or the noise tendency of the image pair is calculated, and the image quality score is higher as the value of the evaluation indicator is higher. As the evaluation indicator, for example, a sum of squared difference of the pixels of the partial image pair, a sum of absolute differences, a normalized cross correlation, a mutual information, a difference or a ratio between SD values, a difference or a ratio between the average tube current values in a case of acquiring the facing partial image pair, or the like can be used. Since these calculation methods are known, the description thereof will be omitted, but for example, the sum of squared difference of the pixels of the facing partial image pair (first image and second image) is calculated by Expression (1).

$$\frac{1}{n} {\sum }_{i = 1}^{n} {( {x}_{i} - {y}_{i} )}^{2} \qquad ( 1 )$$

n: The number of pixels
x: CT value of first image
y: CT value of second image

As the sum of squared difference of the pixels of the facing partial image pair is smaller, the degree of similarity of the images is higher, and the accuracy of the registration performed in the motion correction reconstruction is improved. Therefore, a good image quality of the motion correction reconstruction image is obtained. Therefore, an image quality score S is higher as the sum of squared difference of the pixels is smaller. As a sum of absolute differences or a difference of the tube current value is smaller, the image quality score S is higher.

Further, since as the values of the normalized cross correlation and the mutual information are larger, the accuracy of the motion correction reconstruction is higher, the image quality score S is higher.

As a ratio of the SD value is closer to 1, a noise tendency of a motion image pair is closer, the registration accuracy is improved, and the image quality score is higher. The same applies to the ratio of the tube current value.

Calculation Using Motion information

In step S42, the unidirectional or bidirectional vector distribution information is obtained as the motion information. From the motion information, the total sum of the L1 norms, the total sum of the L2 norms, the total sum of the L∞ norms, or the total sum of the degrees of similarity of cosine can be used as the evaluation indicator, and the image quality score can be calculated using the evaluation indicator. For example, the total sum of the L2 norms of the bidirectional motion information acquired from the facing partial image pair can be calculated by Expression (2).

$$\frac{1}{m} {\sum }_{j = 1}^{m} ( {‖ {v}_{j} ‖}_{2} + {‖ {w}_{j} ‖}_{2} \qquad ( 2 )$$

m: The number of elements of vector
v: First movement vector
w: Second movement vector

As the value is smaller, the motion in this phase is smaller, and the phase is more suitable for the registration. Therefore, the image quality score is calculated such that the image quality score is higher as the total sum of the L2norms is smaller.

### Calculation Using Tomographic Image

As the evaluation indicator using the reconstructed tomographic image, the same indicator as the evaluation indicator calculated from the image pair is used for the front and rear slices. For example, in a case in which the tomographic image is an image reconstructed by applying the motion correction, the sum of squared difference of pixels of continuous slices in a predetermined cardiac phase is used as the evaluation indicator, and since the degree of similarity of the images is higher as the sum of squared difference is smaller, the stable motion correction can be performed, and thus the image quality score has a high value.

The image quality score S can be calculated, for example, by Expressions (3) to (5) in accordance with the correlation between the above-described evaluation indicator and the image quality.

$$S = α ( 1 / Index - a ) \qquad ( 3 )$$

$$S = β * Index - b \qquad ( 4 )$$

$$S = S max - γ ( 1 - Index - c ) \qquad ( 5 )$$

α, β, and γ are any coefficients, Index-a is an evaluation indicator having a negative correlation with the image quality, Index-b is an evaluation indicator having a positive correlation with the image quality, and Index-c is an evaluation indicator that gives the maximum value Smax of the image quality score in a case in which the value is 1.

Although Expressions (3) to (5) are examples in which the image quality score is a linear function of the evaluation indicator or the reciprocal of the evaluation indicator, the calculation expression can also be changed in accordance with the evaluation indicator, and the image quality scores calculated by Expressions (3) to (5) can also be combined to be the total image quality score.

Determination Processing S6 and S60 Using Image Quality Score and Search Phase Re-setting S2 and S23

It is determined whether to return to the processing (FIG. 3: S2, FIG. 5A: S23) of setting the search phase or to determine the optimum phase by using the result of the image quality score. The determination and the search range re-setting processing may be performed by the operator or the apparatus side by displaying a GUI that receives the image quality score and an instruction of the operator on the display device 211 (S60). In a case in which the determination and the search range re-setting processing is performed on the apparatus side, a predetermined threshold value is set in the image quality score, and in a case in which there is a phase in which the calculated image quality score is equal to or higher than the threshold value, the phase having the highest image quality score is set as the optimum phase (S23-1, S23-2). In a case in which a phase having a high image quality score is not obtained, a phase in which the search target phase range calculated in the first time is extended by a certain amount is automatically selected (S23-3).

In a case in which the determination processing is performed by the operator, for example, a GUI that shows a phase range initially set on the display device 211 may be displayed, and the phase inside the two points selected by the operator on the GUI may be manually selected, or a certain range about one point selected by the operator on the same GUI may be semi-automatically selected.

In a case in which it is determined from the result of the determination that the image quality is not sufficient from the image quality score or the like (S6: NO), the processing returns to step S2, a plurality of phases different from the search phase are used as the search range (S23), and the processing from the acquisition (S3) to the presentation (S5) of the tomographic image (without the motion correction) is repeated for the images having the plurality of phases as the center phases.

In the determination processing, in a case in which it is determined that a sufficient image quality is obtained, the operator confirms the image reconstruction phase automatically or through the GUI (S6: YES). In a case in which the operator makes the confirmation, for example, the operator selects one or more series of images of the optimum cardiac phase to terminate the search work for the optimum cardiac phase. Accordingly, the UI controller 233 displays the tomographic image (tomographic image after the motion correction reconstruction) of the optimum cardiac phase which is the center of the searched phases on the display device 211. After repeating the processing, in a case in which a sufficient image quality is obtained, the processing proceeds to step S7 and is terminated in the same manner.

According to the present embodiment, the range in which the optimum phase is searched for is specified, and an arithmetic operation amount related to the image reconstruction is limited, so that it is possible to improve the system responsiveness in the optimum phase search work. In particular, by using the result of applying the motion correction to the image quality evaluation that is the basis of the image quality score, the optimum phase search accuracy in a case in which the motion correction is applied can be improved. In addition, by performing the image reconstruction as the background of the search processing, it is possible to present the optimum cardiac phase image without delay.

### Embodiment 2

The present embodiment is an embodiment related to a function of the UI controller 233 and provides information for supporting the work of setting the image reconstruction phase by the operator. The support information includes, for example, the image quality score of the image in a case in which the motion correction is applied or not applied, and the image (auxiliary information) in a case in which each phase is set as the center phase, and a GUI that receives the setting or change of the search phase range by the operator and a GUI that receives the determination of the optimum phase by the operator are further provided.

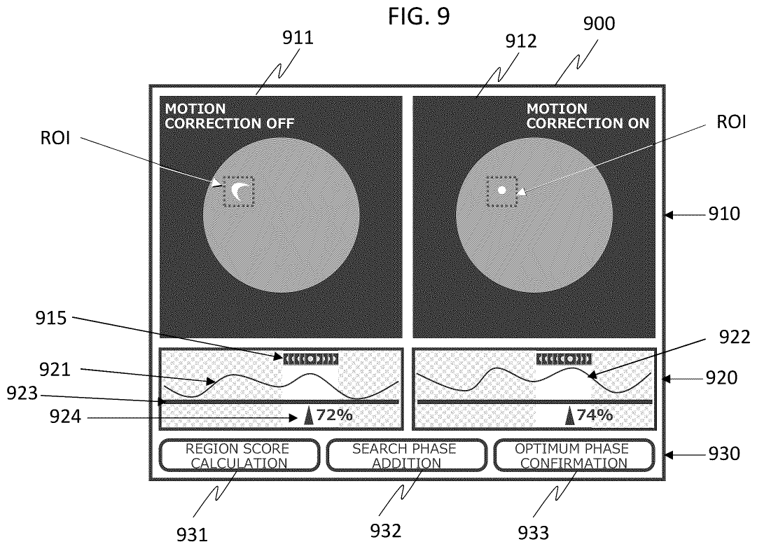

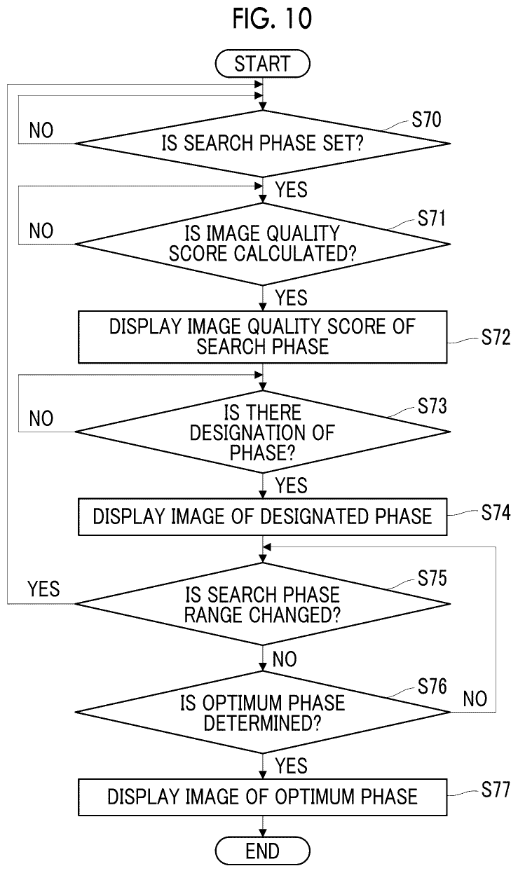

In the present embodiment, it is assumed that the same processing as the processing shown in FIG. 3 or FIG. 7 is performed from the cardiac imaging to the calculation of the image quality score, the description of the processing will be omitted, and the flow of the processing of the UI controller 233 according to the present embodiment will be described with reference to the display screen example of FIG. 9 and the flow of FIG. 10.

As shown in FIG. 9, the display screen is provided with a block (image quality score block) 920 that displays a graph of the image quality score calculated for each phase of the search phase, a block (image display block) 910 that displays the image, and a block (operation button block) 930 that displays buttons for confirming the user instruction, and the blocks 920 and 910 are displayed for a case in which the motion correction is applied and a case in which the motion correction is not applied, respectively.

In the image quality score block 920, the image quality scores are displayed as graphs 921 and 922 with the phase as the horizontal axis, but only the region of the phase in which the image quality score is calculated is explicitly shown in white, and the region of the phase in which the image quality score is not calculated is in a corner. In addition, a cursor (GUI) 924 that can be moved in parallel with a horizontal line 923 indicating the phase is displayed, and the operator can designate any phase by operating the cursor 924. With the movement of the cursor, the image quality score of the position (phase) to which the cursor is moved is enlarged and displayed. In the example shown in FIG. 9, the position of the phase having the highest image quality score in the region in which the image quality score is calculated is selected by the cursor, and the image quality score is displayed as 72% on the left side (motion correction OFF) and 74% on the right side (motion correction ON).

The graphs 921 and 922 showing the image quality scores may show the scores estimated in advance, as the initial state. For example, in S3 and S4 in the first flow in FIG. 3, the score is calculated for all 10 phases sampled at intervals of 10%, and the scores of all phases roughly estimated by the cubic spline interpolation are reflected in the graph in advance.

Images 911 and 912 reconstructed using the phase selected by the cursor as the center phase are displayed in the image display block 910. In a case in which the images 911 and 912 are generated for each phase for which the image quality score is calculated, the images may be displayed as thumbnails 915 and the images may be changed in accordance with the movement of the cursor.

It is further possible to set a region of interest ROI on the images 911 and 912, and in the example shown in the drawing, the region of interest can be designated by surrounding the region of interest with a figure (here, a square) whose size or shape can be changed. In the operation button block 930, GUIs such as a “region score calculation” button 931, a “search phase addition” button 932, and an “optimum phase confirmation” button 933 are displayed, and the operator operates these buttons to execute the corresponding processing in the image processing unit 232.

For example, in a case in which the operator designates the region of interest ROI in the image display block 910 described above and then operates the “region score calculation” button 931, the image quality score calculation unit 322 recalculates the image quality score for the designated region. The result is reflected in the graph of the image quality score block 920 or the image of the image display block 910. In addition, by pressing the “search phase addition” button 932, it is possible to change the region in which the image quality score is calculated (region displayed in white) and the region in which the image quality score is not calculated (corner region), for example, a cursor (not shown) that can move the boundary of the region is displayed, and the search phase range is changed by operating the cursor. In a case in which the image quality score of the search phase is newly calculated after the change, the display of the region division of the image quality score is also changed by reflecting the image quality score.

In a case in which the “optimum phase confirmation” button 933 is operated after the operator checks that a sufficient image quality is obtained in a predetermined phase by using the image quality score or the image in the phase, the optimum phase determination unit 323 determines the optimum phase, and the tomographic image in which the phase is set as the image reconstruction phase is displayed on the image display block 910.

FIG. 10 shows an example of processing of the UI controller 233 that controls the display described above. In a case in which the search phase is set in the search phase setting unit 321 and the image quality score is calculated for each phase, the image quality score in the search phase range is displayed in the image quality score display block (S70 to S72). In a case in which there is the designation of the phase (operation of the cursor 924) by the operator in this display state (S73), the image generated for the designated phase is displayed (S74). In a case in which, in the image quality score calculation processing, a relatively simple image reconstruction is performed, a specific slice may be displayed on the screen by performing high image quality reconstruction similar to normal reconstruction in a case of receiving the designation. Alternatively, a high image quality image may be created separately for the calculation of the image quality score in the background of the calculation of the image quality score, and may be displayed in real time.

In addition, in the display example of FIG. 9, although the image may be generated in both cases in which the motion correction is applied and the motion correction is not applied, only one image may be generated in a case in which only one image is generated, and an instruction to generate the other image may be given via a GUI (not shown) and displayed.

In a case in which the operator checks the displayed image or the image quality score and operates the “search phase addition” button 932 (S75), the search range is changed by the predetermined method as described in the search phase re-setting processing according to Embodiment 1. In a case in which the search phase setting unit 321 receives this change and sets the search phase, the processing returns to step S70, and the processing of S71 to S75 is repeated. In a case in which the “optimum phase confirmation” button 933 is operated without changing the search phase range (S76), the optimum phase determination unit 323 determines the phase designated in step S74 as the optimum phase (S77).

In this way, by graphing the image quality score for each phase or displaying the tomographic image as a thumbnail, the operator can intuitively understand the overall image quality for each phase. It should be noted that the screen example of FIG. 9 and the flow of FIG. 10 are examples, and another GUI that is not presented may be added, or some elements other than the display of the image quality score may be omitted. In addition, as for the display method of the image quality score or the phase, a display format other than the graph can be adopted.

According to the present embodiment, it is possible to provide an interface that can perform the phase search while comparing the motion correction, and thus it is possible to improve the efficiency of the optimum phase search work. Specifically, by providing a GUI that receives an instruction to display the image quality score based on the image quality evaluation taking into account the application/inapplicability of the motion correction, to change the search phase based on the image quality score, or to confirm the optimum phase, it is possible to facilitate the search work of the image reconstruction phase even in a case in which the motion-corrected image reconstruction is executed, and to avoid the complexity of the work or the long time necessary for the work.

### EXPLANATION OF REFERENCES

10: X-ray CT apparatus
20: operation unit
100: gantry
101: bed device
102: X-ray generation device
103: X-ray detector
200: CPU
210: UI unit
211: display device
212: input device
221: storage device
230: arithmetic operation unit
231: image reconstruction unit
232: image processing unit
233: UI controller
321: search phase setting unit
323: optimum phase determination unit
322: image quality score calculation unit
324: partial tomographic image pair generation unit

What is claimed is:

1. An X-ray CT apparatus comprising:
a scanner including an X-ray source and an X-ray detector, which are disposed to face each other, and rotating around a subject; and
an arithmetic operation unit that generates a tomographic image of the subject by using X-ray transmission data detected by the X-ray detector,
wherein the arithmetic operation unit includes
a motion correction reconstruction unit that detects motion information of the subject and performs motion correction reconstruction, and
an image quality score calculation unit that calculates an image quality score for evaluating an image quality in one or a plurality of motion phases with respect to an image reconstructed by the motion correction reconstruction unit.

2. The X-ray CT apparatus according to claim 1, further comprising:
a partial tomographic image pair generation unit that generates a partial tomographic image pair from a pair of projection data in a predetermined angle range about an angle at which rotation angles of the scanner are different by 180 degrees.

3. The X-ray CT apparatus according to claim 2,
wherein the image quality score calculation unit evaluates the image quality of the image after the motion correction reconstruction by using the partial tomographic image pair and calculates the image quality score.

4. The X-ray CT apparatus according to claim 3,
wherein the image quality score calculation unit calculates the image quality score by using at least one of a sum of squared difference of pixels of the partial tomographic image pair, a sum of absolute differences, a normalized cross correlation, a mutual information, or a difference or a ratio between magnitudes of SD values.

5. The X-ray CT apparatus according to claim 3,
wherein the image quality score calculation unit calculates the image quality score by using a difference or a ratio between magnitudes of average tube currents supplied to the X-ray source during capturing of the pair of projection data.

6. The X-ray CT apparatus according to claim 2,
wherein the motion correction reconstruction unit detects the motion by using the partial tomographic image pair generated by the partial tomographic image pair generation unit.

7. The X-ray CT apparatus according to claim 6,
wherein the image quality score calculation unit calculates the image quality score by using the motion information detected by the motion correction reconstruction unit.

8. The X-ray CT apparatus according to claim 1,
wherein the image quality score calculation unit calculates the image quality score by using a tomographic image of an adjacent slice.

9. The X-ray CT apparatus according to claim 1, further comprising:
an optimum phase determination unit that determines an image reconstruction phase by using the motion information of the subject; and
a search phase setting unit that sets a search range for the image reconstruction phase,
wherein the optimum phase determination unit determines the image reconstruction phase by using the image quality score calculated by the image quality score calculation unit for each phase included in the search range.

10. The X-ray CT apparatus according to claim 1, further comprising:
a UI unit that receives user determination for an image reconstruction phase,
wherein the UI unit includes a presentation unit that presents support information including the image quality score calculated by the image quality score calculation unit for a phase in a predetermined range.

11. The X-ray CT apparatus according to claim 10,
wherein the support information includes a tomographic image reconstructed by using a predetermined phase as the image reconstruction phase.

12. An imaging support method of supporting search work for an image reconstruction phase during cardiac imaging using an X-ray CT apparatus, the imaging support method comprising:
a step (1) of setting a phase range in which the image reconstruction phase is searched for by using motion information of a subject; and
a step (5) of searching for the phase range and determining the image reconstruction phase,
wherein, after the step (1),
a step (2) of calculating an image quality score for evaluating an image quality in a case in which motion correction reconstruction is performed for each phase included in the phase range,
a step (3) of presenting the image quality score to an operator, and
a step (4) of receiving determination of the image reconstruction phase from the operator
are included.

13. The imaging support method according to claim 12,
wherein, after the step (2), a step of receiving a change of the phase range in which the search is performed, which is set in the step (1) is further included, and
in the step (2), the image quality score is calculated for the phase range after the change.

14. The imaging support method according to claim 12,
wherein, in the step (2), the image quality score is also calculated for a phase outside the phase range.

15. The imaging support method according to claim 12,
wherein the step (2) includes a step of receiving designation of a region of interest by the operator, and the image quality score is calculated for the region of interest.
